(19) United States
(12) Patent Application Publication
(10) Pub. No.: US 2004/0266997 A1
(43) Pub. Date: Dec. 30, 2004

(54) Degradation products of azithromycin, and methods for their indentification

(76) Inventors: Michael Pesachovich, Givat-Shmuel (IL); Sarah Isaacs, Hertzelia (IL); Claude Singer, Kfar Saba (IL); Eduard Schwartz, Rehovot (IL); Edit Berger, Tel-Aviv (IL)

Correspondence Address: KENYON & KENYON, ONE BROADWAY, NEW YORK, NY, 10004, US

(21) Appl. No.: 10/809,065

(22) Filed: Mar. 25, 2004

Related U.S. Application Data
(60) Provisional application No. 60/457,846, filed on Mar. 25, 2003.
(60) Provisional application No. 60/458,186, filed on Mar. 26, 2003.

(51) Int. Cl. A61K 31/7052; C07H 17/08

(52) U.S. Cl. 536/7.4; 514/28

(57) ABSTRACT

The invention is directed to degradation products of azithromycin, methods for the preparation and identification of the degradation products which may be produced during storage and/or synthesis of azithromycin.

### RELATED APPLICATIONS

This application claims the benefit of U.S. Provisional Application Ser. Nos. 60/457,846 filed Mar. 25, 2003, and 60/458,186 filed Mar. 26, 2003, both of which are incorporated herein.

### FIELD OF THE INVENTION

The invention encompasses the degradation products of azithromycin which may be produced during synthesis and storage of azithromycin and to methods of identifying such degradation products. The present invention also encompasses the compounds useful as reference markers for the analysis of azithromycin and pharmaceutical formulations thereof.

### BACKGROUND OF THE INVENTION

Azithromycin has the chemical name [2R-(2R*,3S*,4R*,5R*,8R*,10R*,11R*,12S*,13S*,14R*)]-13-[(2,6-dideoxy-3C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one and the following chemical structure:

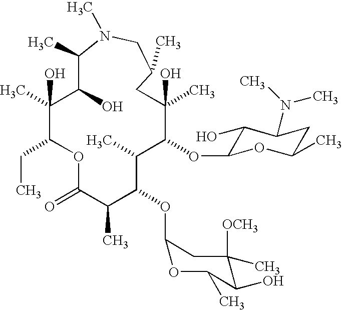

Azithromycin is one of the macrolide antibiotics, so named because they contain a many-membered lactone ring to which are attached one or more deoxy sugars. Other macrolid antibiotics include erythromycin and clarithromycin. Azithromycin and the other macrolid antibiotics are bacteriostatic agents which act by binding to the 50S ribosomal subunit of susceptible microorganisms, and thus interfering with microbial protein synthesis.

Macrolide antibiotics of the erythromycin class, such as erythromycin A, are known to be unstable in an acidic environment and are inactivated by gastric acids. See, Goodman and Gilman's, The Pharmacological Basis of Therapeutics, p. 1137 (Joel G. Hardman et al., eds. 9th Ed. 1996); Vinckier et al., Int. J. Pharmaceutics, 55, 67-76 (1989); Cachet et al., Int. J. Pharmaceutics, 55, 59-65 (1989); Fiese et al., J. Antimicrobial Chemother., 25 (suppl.A) 39-47 (1990).

Azithromycin is a semi-synthetic antibiotic which differs chemically from erythromycin in that a methyl-substituted nitrogen atom is incorporated into the lactone ring. The replacement of the keto group in the lactone ring with the N-methyl group in the lactone ring improves the stability of azithromycin over erythromycin in an acidic environment. U.S. Pat. Nos. 4,517,359 and 4,474,768 disclose processes for the preparation of azithromycin and the use of azithromycin as an antibiotic and are incorporated herein by reference.

Azithromycin is subject to degradation that may occur during manufacture and/or storage. For example, azithromycin is susceptible to degradation if exposed to elevated temperatures and/or air during manufacturing processes, processes that include formulation of the pharmaceutical dosage form. One particular example of oxidative degradation is the oxidation of the exocyclic amine group of azithromycin. The azithromycin susceptibility to degradation leads to deviation of the drug product from regulatory purity requirements even prior to the product reaching the patient. In addition, once formulated, azithromycin tends to degrade under normal storage conditions, which may result in the presence of unacceptable levels of impurities at the time of administration.

Therefore, a continuing need exists to identify the degradation products and to develop readily usable identification methods to determine azithromycin degradation products.

### SUMMARY OF THE INVENTION

An embodiment of the invention encompasses methods for the detection and identification of azithromycin degradation products and novel intermediates thereof. Another embodiment of the invention encompasses azithromycin degradation products, including, but not limited to, the azithromycin degradation product identified by an HPLC relative retention time of 0.22, 0.26, or 0.80. The azithromycin degradation product identified by a HPLC relative retention time of 0.22, has substantially the following structure I:

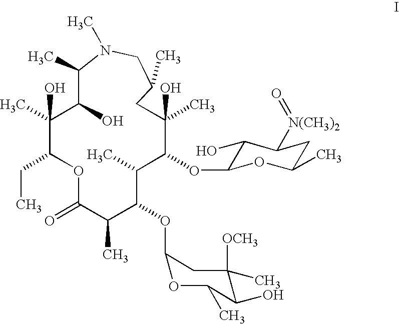

Another embodiment of the invention encompasses the azithromycin degradation product identified by a relative retention time of 0.26 and having substantially structure II:

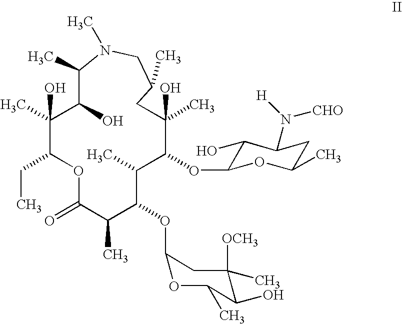

Yet another embodiment of the invention encompasses the azithromycin degradation product identified by a relative retention time of 0.80 and having substantially structure III:

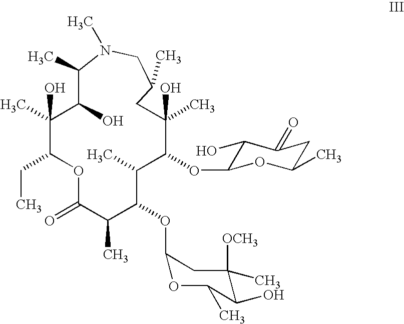

Yet another embodiment of the invention encompasses methods for the isolation of azithromycin degradation products including, but not limited to, degradation products identified by an HPLC relative retention time of 0.22, 0.26 and 0.80. Another embodiment of the invention encompasses azithromycin having less than about 0.5% by weight of at least one degradation product having a relative retention time on an HPLC relative to azithromycin of 0.22, 0.26, or 0.80, preferably, less than about 0.3% by weight of at least one degradation product having a relative retention time on an HPLC relative to azithromycin of 0.22, 0.26, or 0.80 as calculated against azithromycin standard.

Another embodiment of the invention encompasses methods to analyze azithromycin purity comprising assaying azithromycin to determine the presence and an amount, if any, of azithromycin degradation products. Yet another embodiment of the invention encompasses methods to determine azithromycin stability comprising assaying azithromycin to determine the presence and amount, if any, of azithromycin degradation products. Yet another embodiment of the invention encompasses methods to analyze azithromycin purity, stability to degradation, or both comprising assaying a sample of azithromycin by HPLC, and determining the presence and/or amount of azithromycin degradation products identified by an HPLC relative retention time of 0.22, 0.26, or 0.80.

### BRIEF DESCRIPTION OF THE FIGURES

FIG. 1 illustrates an HPLC chromatogram of a sample of degraded azithromycin having azithromycin degradation products, which were identified.

FIG. 2 illustrates an HPLC chromatogram of an azithromycin degradation product having a relative retention time (RRT) of 0.26.

FIG. 3 illustrates a MS of an azithromycin degradation product having a RRT of 0.26.

FIG. 4 illustrates an HPLC chromatogram of an enriched sample of azithromycin degradation products.

FIG. 5 illustrates an HPLC chromatogram of an azithromycin degradation product having a RRT of 0.22.

FIG. 6 illustrates the UV spectrum of azithromycin degradation product having a RRT of 0.22.

FIG. 7 illustrates an HPLC chromatogram of an azithromycin degradation product having a RRT of 0.26.

FIG. 8 illustrates the UV spectrum of azithromycin degradation product having a RRT of 0.26.

FIG. 9 illustrates an HPLC chromatogram of an azithromycin degradation product having a RRT of 0.80.

FIG. 10 illustrates the UV spectrum of azithromycin degradation product having a RRT of 0.80.

### DETAILED DESCRIPTION OF THE INVENTION

Definitions

As used herein, the term “AZT” refers to azithromycin. The term “DMAZT” refers to azaerythromycin A (USP), desmethyl azithromycin. The term “TAZT” refers to tosyl azithromycin. The term “BH” refers to butylated hydroxyanisole. The term “BHT” refers to butylated hydroxytoluene. The term “PG” refers to propyl gallate. The term “PVP” refers to polyvinylpyrrolidone. The term “SLS” refers to sodium lauryl sulfate. The term “API” refers to active pharmaceutical ingredient. The term “LOD” refers to loss on dry.

As used herein, unless otherwise indicated, the term “azithromycin” includes, but is not limited to, azithromycin salts, including hydrochloride salts; solvates, including hydrates, alcoholates, and esters; and physiologically functional derivatives thereof. The term “azithromycin” also includes all polymorphous forms.

As used herein, the term “relative response factor” refers to the ratio of the absorbency between two compounds as a predetermined wavelength.

As used herein, the term “unit dosage form” refers to the amount of azithromycin, or a derivative thereof, which is effective to produce a therapeutic effect in a subject.

Description of the Invention

Azithromycin is unstable and prone to produce degradation products upon manufacture and/or storage. Not to be bound by theory, it is believed that one degradation pathway is the oxidation of azithromycin in the presence of oxidizing agents, such as atmospheric oxygen. The invention encompasses methods of isolating and identifying the degradation products of azithromycin. During azithromycin synthesis and storage, the degradation products may be isolated using chromatography, thus allowing for purity levels wherein the structural determination of the degradation products is feasible.

The synthesis of azithromycin typically commences by the fermentation of erythromycin A. In a subsequent synthetic step, a methyl-substituted nitrogen atom is incorporated into the lactone ring of erythromycin A to form azithromycin. The process combines a natural fermentation step with a synthetic step, thus creating a semi-synthetic synthesis. Generally, products made by semi-synthetic synthesis are of lower purity and have a greater quantity and variety of impurities as compared to products of completely synthetic processes.

The invention encompasses analytical methods to determine the purity and/or the degradation stability of azithromycin comprising assaying an amount of azithromycin; determining the presence of degradation products; identifying the degradation products; and quantifying the amount of degradation products. More particularly, the present invention encompasses analytical methods to determine the purity and/or stability to degradation of azithromycin by assaying an amount of azithromycin, and determining the presence of azithromycin degradation products identified by an HPLC relative retention time of 0.22, 0.26, or 0.80. Thus, the invention also encompasses azithromycin degradation products identified by an HPLC relative retention time of 0.22, 0.26, or 0.80.

A method of the invention for the isolation of azithromycin degradation products comprises obtaining an azithromycin sample; isolating at least one azithromycin degradation product using chromatography, and identifying the azithromycin degradation product. The skilled artisan can easily determine the amount of azithromycin necessary to perform the isolation. The method may further comprise quantifying the azithromycin degradation product.

The chromatography used in the methods of the invention include, but are not limited to, thin layer chromatography, column chromatography, flash chromatography, or high pressure liquid chromatography (HPLC). Typically, the degradation products were isolated by using HPLC, MS, or both.

Typically, the HPLC is performed using a column of 150×4.6 mm, packing material of Kromasil KR 100-5C18, 5μ and an eluent of 40% 0.05 M K₂HPO₄ adjusted to a pH of 8.2 and 60% acetonitrile. The flow rate may be 0.9 ml/min, the detector set at 210 nm, and column temperature about 30° C. The column packing material of the HPLC may be a C8-C18 including packing embedded with polar groups and particles in the size of about 3μ to 10μ. Preferably, the packing materials is C18, 5μ, silica, such as Kromasil KR 100-5C18 sold by Eka Chemicals, Separation Products (SE-445 80 Bohus, Sweden). Any suitable column may be used, preferably a 150×4.6 mm column.

Preferably, the degradation products are isolated by HPLC using any suitable eluent including, but are not limited to, acetonitrile, dipotassium hydrogen phosphate (K₂HPO₄), ammonium acetate, ammonium formate, carbonate salts, ammonium hydroxide, and combinations thereof. Carbonate salts include, but are not limited to, sodium, potassium, calcium, or magnesium salts of carbonate or bicarbonate. In one preferred embodiment, the eluent mixture comprises acetonitrile in about 40% or greater v/v of the solvent mixture, and more preferably, acetonitrile comprises about 60% v/v of the solvent mixture. Optionally, the eluent may contain at least one buffer salt wherein the cation is sodium, potassium, or ammonium and the anion is phosphate, acetate, formate, or carbonate. For example, one buffer is 0.05 M K₂HPO₄. During HPLC chromatography, typically the pH is in the range of about 7.5 to about 10, and preferably, the pH is about 8.2. The temperature of the column is maintained from about 20° C. to about 50° C., and more preferably, the temperature is about 30° C. Typically, the flow rate used during HPLC isolation is about 0.5 ml/min to about 2 ml/min, and preferably about 0.9 ml/min.

Identification of the degradation products may be performed using at least one of nuclear magnetic resolution (NMR), HPLC, infrared (IR), ultra violet absorption (UV), or mass spectrometry (MS). For example, the degradation products may be identified using HPLC-MS/NMR. Typically, the degradation products were identified using a combined HPLC and MS analysis, such as API-300 Sciex, HPLC Perkin-Elmer 200, Autosampler Perkin-elmer 200. Typically, identification by HPLC uses the above described parameters for isolation of azithromycin degradation products. Typically, the MS is performed by using triple-Q HPLC/MS analysis.

Using the HPLC-MS methodology described above, the degradation products of AZT were determined based on an HPLC relative retention times as relative to azithromycin The degradation products were identified by relative retention times of 0.22, 0.26, or 0.80.

The azithromycin degradation product having a relative retention time of 0.22 may also be identified by the following chemical structure (I):

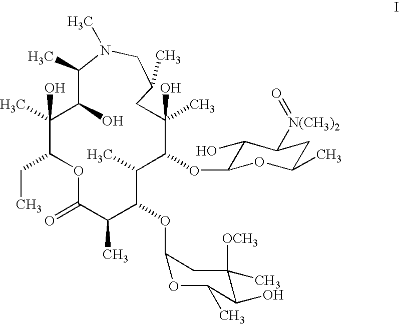

The azithromycin degradation product having a relative retention time of 0.26 may also be identified by the following chemical structure (II):

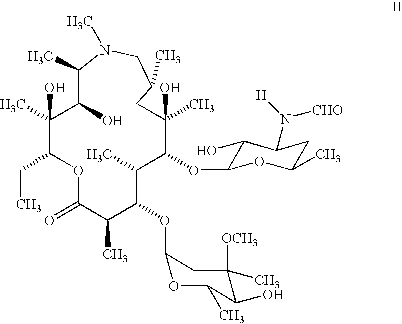

The azithromycin degradation product identified as having a relative retention time of 0.80 may also be identified by the following chemical structure (III):

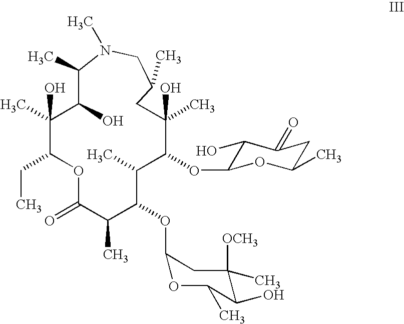

Another embodiment of the invention encompasses azithromycin containing less than about 0.5% by weight of degradation products of at least one of structure I, II, or III. Preferably, the azithromycin contains less than about 0.3% by weight of at least one degradation product of structure I, II, or III.

Having described the invention with reference to certain preferred embodiments, other embodiments will become apparent to one skilled in the art from consideration of the specification. The invention is further defined by reference to the following examples describing in detail the identification, isolation, and purification methods of the invention. It will be apparent to those skilled in the art that many modifications, both to materials and methods, may be practiced without departing from the scope of the invention.

### EXAMPLES

Although the following examples illustrate the practice of the present invention in some of its embodiments, the examples should not be construed as limiting the scope of the invention.

### Example 1

### Azithromycin Analysis Using HPLC

After degradation, a sample of azithromycin was studied using HPLC to determine the level of impurities within the sample. The azithromycin was degraded by heating the azithromycin to at most 55° C. for 2 months. The analytical conditions of the HPLC were column of 150×4.6 mm, packing material of Kromasil KR 100-5C18, 5μ and an eluent of 40% 0.05 M K₂HPO₄ adjusted to a pH of 8.2 and 60% acetonitrile. The flow rate was 0.9 ml/min, the detector set at 210 nm, and column temperature was 30° C. The samples were injected into the HPLC, and after 35 minutes, the sample was studied. The impurities were determined by their relative retention times as compared to azithromycin and were found to have the relative retention times (RRT) of: 0.22, 0.26, 0.34, 0.37, 0.40, 0.80, 1.53, and 1.63.

### Example 2

### Identification of Azithromycin Degradation Products

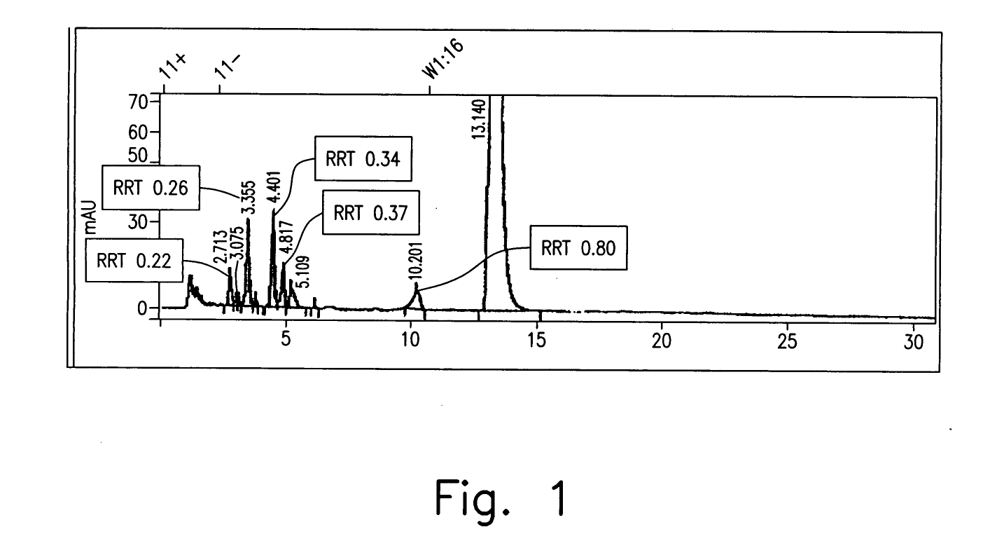

A sample of azithromycin was allowed to degrade as described in Example 1. Thereafter, the sample was analyzed at a concentration of 7 mg/ml by HPLC as described in Example 1. Degradation peaks were found to have relative retention times at 0.22, 0.26, 0.34, 0.37, and 0.80 as compared to azithromycin. HPLC analysis of azithromycin before and after degradation allowed for the identification of the degradation products. See FIG. 1.

Example 3: Method 1 for the Isolation of the Degradation Products

A sample of azithromycin was allowed to degrade at 55° C. for three months. Thereafter, the sample was subjected to flash chromatography using a column packed with RP-18 10 μm, and a stepwise solvent gradient of acetonitrile:ammonium hydroxide:water with increasing eluting force, which was achieved by acetonitrile. The conditions necessary for flash chromatography were determined using thin layer chromatography (TLC). RP-18 TLC was effected using acetonitrile:ammonium hydroxide:water as the eluent in a ratio of 7:1:2 and 8:1:1 and acetonitrile:ammonium in a ratio of 9:1. The degradation products were enriched using flash chromatography, on a Lichrosphere RP-18 10 μm column, with stepwise solvent gradient of acetonitrile:NH₄OH:water with increasing eluting force, which was achieved by acetonitrile. Impurities from the enriched fractions were isolated by semipreparative chromatography on Kromasil KR-100 RP-18 20 cm*10 cm, 10 μm with eluent 50% 0.05M of dipotassium hydrogen phosphate (K₂HPO₄) adjusted to pH 8.2 and 50% acetonitrile. Fractions were collected and extracted into dichloromethane. The organic layer was washed with diluted ammonia solution and evaporated to dryness.

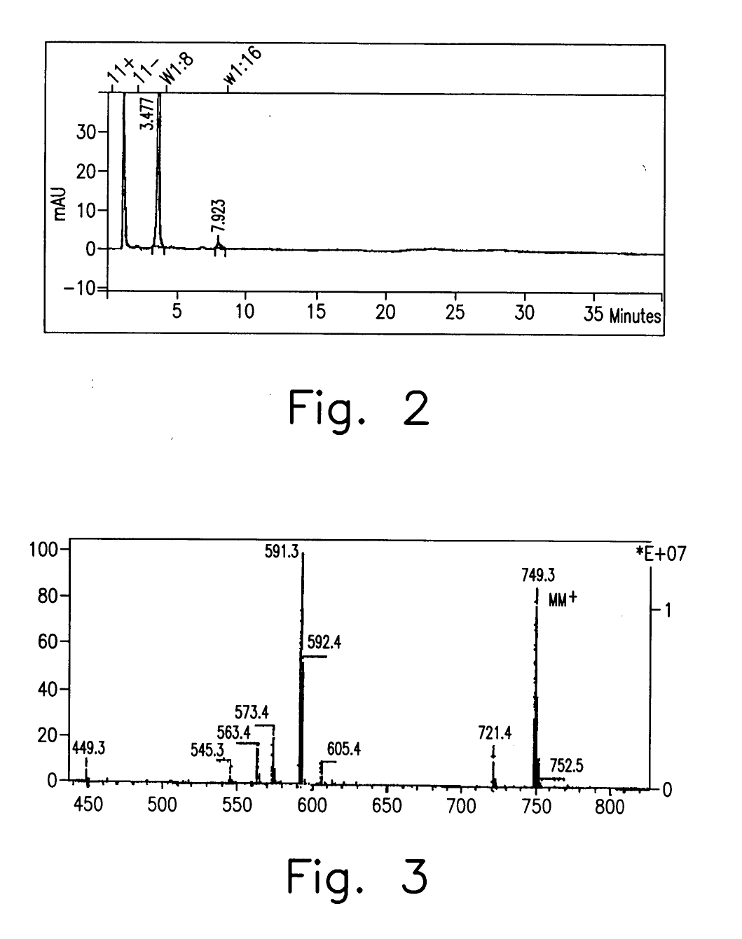

The HPLC chromatogram for the degradation product RRT 0.26 is illustrated in FIG. 2. The mass spectrum of the isolated impurity RRT 0.26 is illustrated in FIG. 3.

### Example 4

### Method 2 for Isolation of the Degradation Products

Azithromycin (20 g) was dissolved in acetonitrile (100 ml) containing NH₄OH (224 μl, 25%), and extracted with n-hexane (13 ×200 ml). The acetonitrile phase was separated and evaporated to dryness in a vacuum distillation unit. The remaining solids (4 g) were redissolved in 50% aqueous acetonitrile and allowed to stand. After three days, AZT precipitated from a yellow supernatant. The AZT was collected by filtration using a 0.45 μm pore size membrane filter. The enriched samples (2.5 ml) were successively injected onto a Waters X-Terra MS C₁₈ column (19×300 mm) and eluted a 5 ml/min at room temperature with a step-like gradient solvent system as described below. The acetonitrile was evaporated under reduced pressure and the remaining aqueous suspensions were frozen and lyophilised.

| Time (min) | Eluent |
|---|---|
| 0 | 10 mM NH₃ in 48% aqueous acetonitrile (ACN) |
| 60 | 10 mM NH₃ in 48% aqueous ACN:ACN (6:4 v/v) |
| 85 | ACN |
| 100 | 10 mM NH₃ in 48% aqueous ACN |

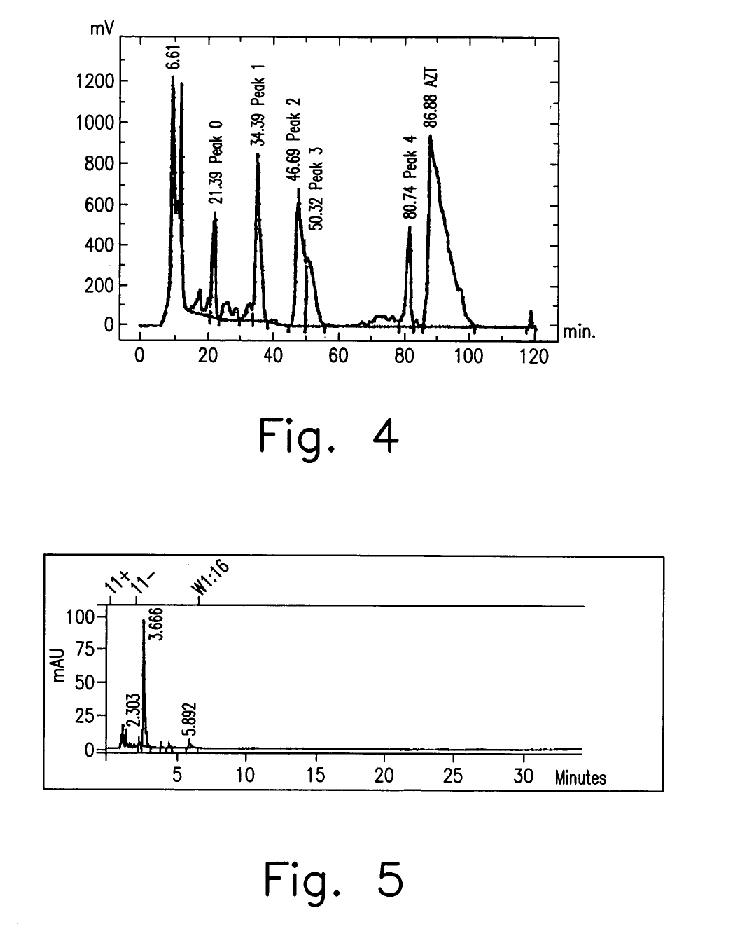

FIG. 4 illustrates the HPLC chromatogram of the enriched sample, the degradation products are identified as peak 0, peak 1, peak 2, peak 3, and peak 4.

Table 1 illustrates the impurities obtained from the degraded azithromycin, the retention time using HPLC, formula, and identification of the fragments by MS.

TABLE 1
Azithromycin Degradation Products

| RT (HPLC MS) | RRT (HPLC) | Formula 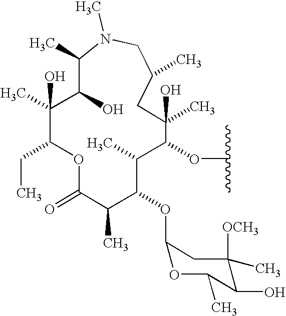 | a) name (pharmaeuropa) b) mz + 1 c) current status and name | Fragments |
|---|---|---|---|---|
| 18 | 1.0 | 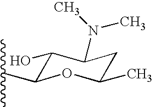 | a) azithromycin b) 749.5 | [749.5 decladinosyl + H] =591.6 [591.6 −desosaminyl + H] =434.1 |
| 3.5 | 0.22 | 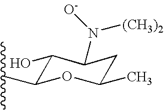 | a) b) 765.5 c) isolated, potential impurity AZT-N- oxide | [765.6 −decladinosyl + H] =607.5 [607.5 −desosoaminyl-N- oxide + H] = 434.5 |
| 4.5 | 0.26 | 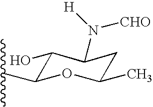 | a) b) 749.5 c) isolated, potential impurity N-formyl- N-di(demethyl)- AZT | [749.5 −decladisonyl + H] =591.6 [591.6 − N-formyl- desososaminyl + H] =434.1 |
| 18.5 | 0.80 | 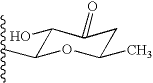 | a) b) 720.5 c) isolated, potential impurity, desdimethyl-keto- AZT | [720.6 −decladinosyl + H] =562.4 |

### Example 5

### UV, NMR, and MS Spectroscopy

The UV spectra of the isolated impurities were evaluated using Photo Diode Array (PDA 996 Waters) in the range of 200-350 nm attached to HPLC Waters Allians HPLC system and running the isolated impurities and degraded sample under the HPLC conditions described in Example 1.

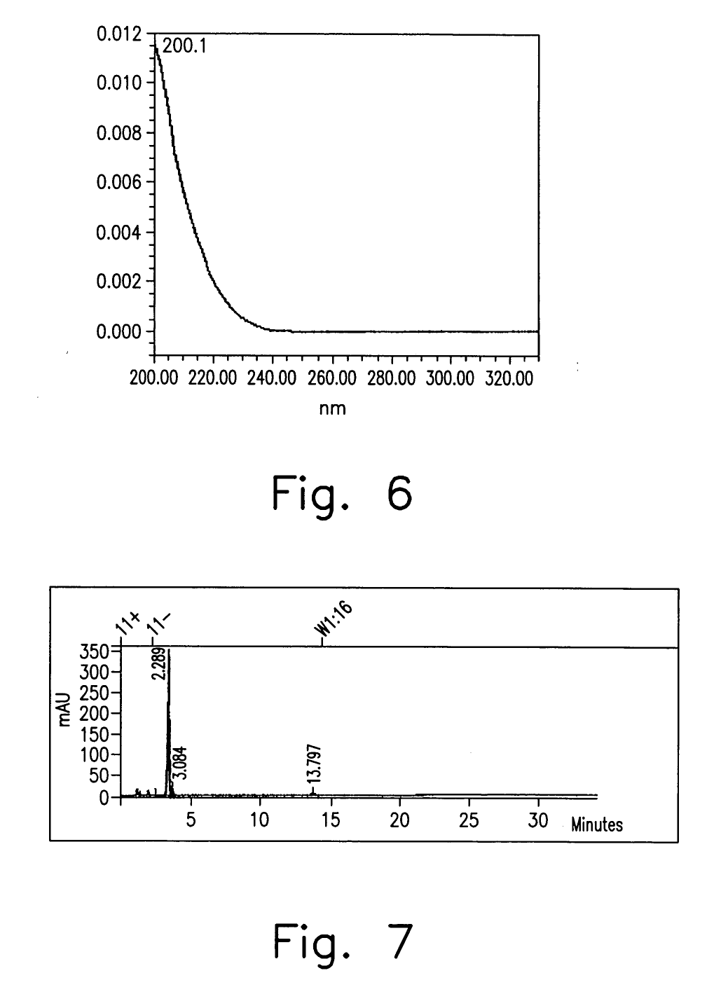

The isolated impurity samples of Examples 3 and 4 were characterized using UV spectroscopy. FIG. 5 illustrates the HPLC spectrum and FIG. 6 illustrates the UV spectrum of azithromycin degradation product having a relative retention time of 0.22. The ¹H NMR and ¹³C NMR were taken for the azithromycin degradation product having a relative retention time of 0.22, Table 1 summarizes the data. The compound having a RRT of 0.22 was identified as azithromycin having a side chain of the following structure:

TABLE 1
¹H and ¹³C NMR data of Impurity with RRT of 0.22

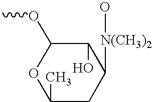

| Position # | δ_{C} | | δ_{H} | |
|---|---|---|---|---|
| 1 | 179.1 | s | | |
| 2 | 45.4 | d | 2.72 | dq |
| 2-Me | 14.5 | q | 1.19 | d |
| 3 | 77.8 | d | 4.26 | brt |
| 4 | 42.7 | d | 1.98 | m |
| 4-Me | 8.7 | q | 1.08 | d |
| 5 | 83.6 | d | 3.63 | d |
| 6 | 73.7 | s | | |
| 6-Me | 27.6 | q | 1.31 | s |
| 7 | 42.2 | t | 1.77 | d |
| | | | 1.32 | m |
| 8 | 26.7 | d | 2.02 | m |
| 8-Me | 22.0 | q | 0.91 | d |
| 9 | 70.0 | t | 2.53 | d |
| | | | 2.03 | t |
| 9-NMe | 36.0 | q | 2.32 | s |
| 10 | 62.6 | d | 2.69 | dq |
| 10-Me | 7.0 | q | 1.09 | d |
| 11 | 73.4 | d | 3.67 | brs |
| 12 | 74.1 | s | | |
| 12-Me | 16.3 | q | 1.10 | s |
| 13 | 77.5 | d | 4.71 | dd |
| 14 | 21.3 | t | 1.91 | m |
| | | | 1.45 | m |
| 14-Me | 11.2 | q | 0.89 | t |
| 1′ | 102.4 | d | 4.54 | d |
| 2′ | 72.4 | d | 3.78 | dd |
| 3′ | 76.7 | d | 3.37 | ddd |
| 3′-NMe₂ | 52.1 | q | 3.20 | s |
| | 58.9 | q | | |
| 4′ | 34.9 | t | 1.99 | m |
| | | | 1.34 | q |
| 5′ | 66.9 | d | 3.65 | m |
| 6′ | 21.6 | q | 1.27 | d |
| 1″ | 94.4 | d | 5.19 | d |
| 2″ | 34.6 | t | 2.38 | d |
| | | | 1.59 | dd |
| 3″ | 73.0 | s | | |
| 3″-Me | 21.1 | q | 1.25 | s |
| 3″-OMe | 49.7 | q | 3.39 | s |
| 4″ | 78.1 | d | 3.06 | brd |
| 5″ | 65.6 | d | 4.08 | dq |
| 6″ | 18.2 | q | 1.33 | d |

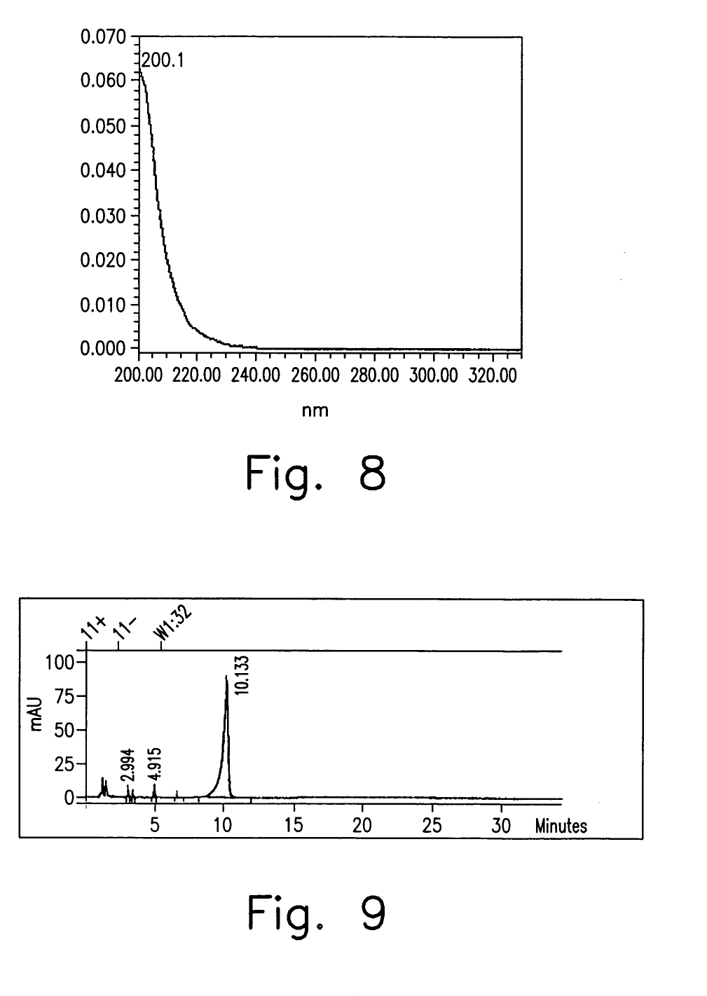

The mass spectroscopy used a (+) FAB MS an provided peaks at m/z: 771(Mna⁺), 749 (MH⁺), 633, 590, 573, 416, 414, 374, 198, 186, and 149. See FIG. 8.

FIG. 7 illustrates the HPLC spectrum and FIG. 8 illustrates the UV spectrum of azithromycin degradation product having a relative retention time of 0.26. The ¹H NMR and ¹³C NMR were taken for the azithromycin degradation product having a relative retention time of 0.26, Table 2 summarizes the data. The compound having a RRT of 0.26 was identified as an azithromycin degradation product, wherein azithromycin has a side chain of the following formula:

TABLE 2
¹H and ¹³C NMR data of Impurity with RRT of 0.26 in CDCl₃

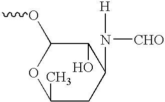

| Position # | δ_{C} | | δ_{H} | |
|---|---|---|---|---|
| 1 | 178.6 | s | | |
| 2 | 45.4 | d | 2.72 | m |
| 2-Me | 14.7 | q | 1.19 | d |
| 3 | 77.9 | d | 4.23 | brs |
| 4 | 42.1 | d | 1.99 | m |
| 4-Me | 9.4 | q | 0.99 | d |
| 5 | 84.0 | d | 3.62 | m |
| 6 | 73.5 | s | | |
| 6-Me | 27.3 | q | 1.31 | s |
| 7 | 42.3 | t | 1.69 | d |
| | | | 1.26 | m |
| 8 | 26.6 | d | 2.03 | m |
| 8-Me | 22.0 | q | 0.92 | brd |
| 9 | 69.8 | t | 2.57 | d |
| | | | 2.13 | m |
| 9-NMe | 36.2 | | 2.36 | brs |
| 10 | 62.8 | d | 2.73 | m |
| 10-Me | 6.8 | q | 1.12 | brd |
| 11 | 73.4 | d | 3.66 | brs |
| 12 | 74.2 | s | | |
| 12-Me | 16.3 | q | 1.09 | s |
| 13 | 77.5 | d | 4.73 | m |
| 14 | 21.3 | t | 1.90 | m |
| | | | 1.45 | m |
| 14-Me | 11.2 | q | 0.89 | t |
| 1′ | 102.5 | d | 4.49 | d* |
| 2′ | 74.5 | d | 3.27 | dd** |
| 3′ | 50.6 | d | 3.93 | dddd |
| 3′-NH | | | 5.95 | brs*** |
| 3′NC(O)H | 162.0 | d | 8.22 | s**** |
| 4′ | 38.6 | t | 2.13 | m |
| | | | 1.31 | m |
| 5′ | 68.2 | d | 3.64 | m |
| 6′ | 20.8 | q | 1.21 | d |
| 1″ | 94.7 | d | 5.10 | brs |
| 2″ | 34.7 | t | 2.34 | d |
| | | | 1.58 | dd |
| 3″ | 73.0 | s | | |
| 3″-Me | 21.6 | q | 1.25 | s |
| 3″-OMe | 49.5 | q | 3.33 | s |
| 4″ | 78.0 | d | 3.05 | brs |
| 5″ | 65.8 | d | 4.08 | dq |
| 6″ | 18.1 | q | 1.32 | d |

The mass spectroscopy used a (+) FAB MS an provided peaks at m/z: 771(MNa⁺), 749 (MH⁺), 633, 591, 573, 416, 374, 198, and 186.

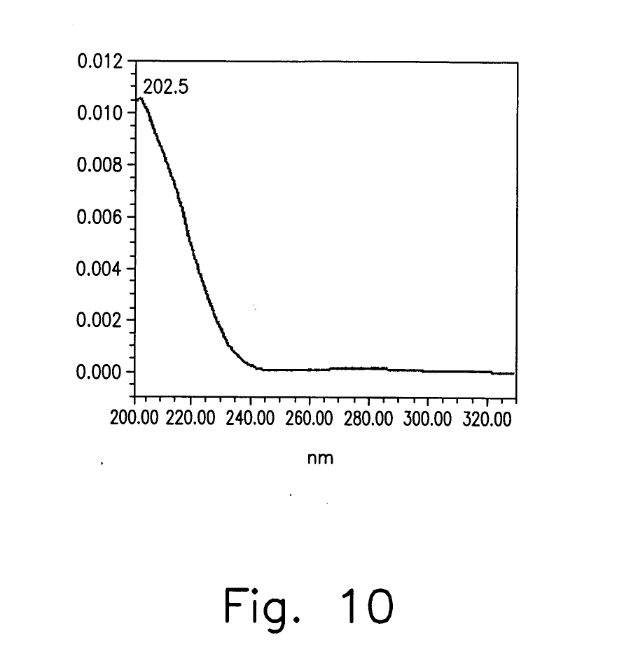

FIG. 9 illustrates the HPLC spectrum and FIG. 10 illustrates the UV spectrum of azithromycin degradation product having a relative retention time of 0.80. The ¹H NMR and ¹³C NMR were taken for the azithromycin degradation product having a relative retention time of 0.80, Table 3 summarizes the data. The compound having a RRT of 0.80 was identified as an azithromycin degradation product, wherein azithromycin has a side chain of the following formula:

TABLE 3
¹H ¹³C NMR data of Impurity with RRT of 0.80 in CDCl₃ and CD₃OD

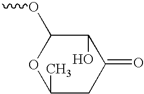

| Position # | δ_{C} | | δ_{H} | |
|---|---|---|---|---|
| 1 | 178.2 | s | | |
| 2 | 45.1 | d | 2.60 | m |
| 2-Me | 14.4 | q | 1.04 | d |
| 3 | 77.9 | d | 4.06 | brd |
| 4 | 42.0 | d | 1.84 | m |
| 4-Me | 8.5 | q | 0.86 | d |
| 5 | 84.2 | d | 3.52 | d |
| 6 | 73.5 | s | | |
| 6-Me | 27.1 | q | 1.24 | s |
| 7 | 42.0 | t | 1.64 | d |
| | | | 1.19 | m |
| 8 | 26.4 | d | 1.88 | m |
| 8-Me | 21.6 | q | 0.80 | d |
| 9 | 69.7 | t | 2.44 | d |
| | | | 1.98 | t |
| 9-NMe | 35.8 | q | 2.18 | s |
| 10 | 62.5 | d | 2.60 | m |
| 10-Me | 6.7 | q | 0.96 | d |
| 11 | 73.5 | d | 3.47 | brs |
| 12 | 74.2 | s | | |
| 12-Me | 16.0 | q | 0.97 | s |
| 13 | 76.7 | d | 4.60 | dd |
| 14 | 21.1 | t | 1.74 | m |
| | | | 1.33 | m |
| 14-Me | 10.8 | q | 0.76 | t |
| 1′ | 103.0 | d | 4.43 | d |
| 2′ | 78.4 | d | 3.90 | d |
| 3′ | 206.4 | s | | |
| 4′ | 47.2 | t | 2.40 | dd |
| | | | 2.28 | dd |
| 5′ | 67.0 | d | 3.67 | ddq |
| 6′ | 21.1 | q | 1.20 | d |
| 3″ | 72.9 | s | | |
| 3″-Me | 21.1 | q | 1.08 | s |
| 3″-OMe | 48.8 | q | 3.07 | s |
| 4″ | 77.8 | d | 3.89 | dd |
| 5″ | 65.2 | d | 3.91 | m |
| 6″ | 17.8 | q | 1.17 | d |

The mass spectroscopy used a (+) FAB MS an provided peaks at m/z: 743(MNa⁺), 721 (MH⁺), 704, 590, 574, 544, 416, 374, 272, 198, and 186.

What is claimed is:

1. An azithromycin degradation product identified by an HPLC relative retention time of 0.22, 0.26, or 0.80.

2. An azithromycin degradation product identified by a HPLC relative retention time of 0.22 having substantially the following structure I:

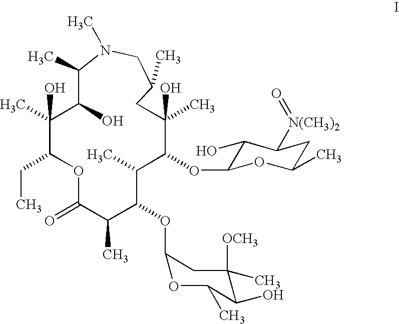

3. An azithromycin degradation product identified by a HPLC relative retention time of 0.26 having substantially the following structure II:

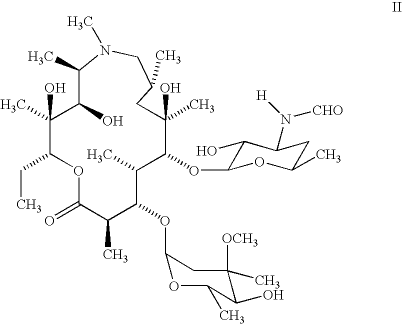

4. An azithromycin degradation product identified by a HPLC relative retention time of 0.80 and having the following structure III:

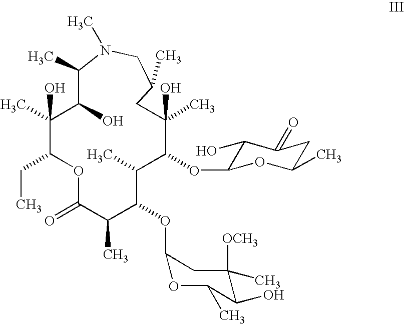

5. Azithromycin comprising less than about 0.5% by weight of at least one degradation product having a relative retention time on an HPLC relative to azithromycin of 0.22, 0.26, or 0.80.

6. The azithromycin according to claim 5, having less than about 0.3% by weight of at least one degradation product having a relative retention time on an HPLC relative to azithromycin of 0.22, 0.26, or 0.80.

7. A method to analyze azithromycin purity comprising:
assaying azithromycin using an HPLC to determine the presence of azithromycin degradation products;
identifying azithromycin degradation products; and
quantifying the azithromycin degradation products.

8. The method according to claim 7, wherein the identification step comprises searching and identifying on the HPLC spectrum azithromycin degradation products having a relative retention time of about 0.22, 0.26, and 0.80.

9. A method to determine azithromycin stability comprising:
assaying azithromycin using HPLC to determine the presence of azithromycin degradation products;
identifying the azithromycin degradation products; and
quantifying the azithromycin degradation products.

10. The method according to claim 9, wherein the identification step comprises searching and identifying on the HPLC spectrum azithromycin degradation products having a relative retention time of about 0.22, 0.26, and 0.80.
